(19) United States
(12) Patent Application Publication
(10) Pub. No.: US 2009/0162303 A1
(43) Pub. Date: Jun. 25, 2009

(54) COMPOSITION CONTAINING TWO POLYESTERS

(75) Inventors: Claudia Barba, Paris (FR); Chrystel Paute, Saclay (FR); Audrey Ricard, Paris (FR); Pascal Giustiniani, La Garenne Colombes (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 12/142,256

(22) Filed: Jun. 19, 2008

Related U.S. Application Data
(60) Provisional application No. 60/929,739, filed on Jul. 11, 2007.

(30) Foreign Application Priority Data
Jun. 21, 2007 (FR) 07 55935

(51) Int. Cl. A61K 31/765; A61Q 3/00; A61Q 1/02; A61Q 5/00; A61Q 19/00

(52) U.S. Cl. 424/61; 424/78.03; 424/70.11; 424/63

(57) ABSTRACT

Composition containing two polyesters. Treatment method employing the composition and use of this composition for caring for or making up the skin or lips. Novel polyesters.

### REFERENCE TO PRIOR APPLICATIONS

This application claims priority to U.S. provisional application 60/929,739 filed Jul. 11, 2007, and to French patent application 0755935 filed Jun. 21, 2007, both incorporated herein by reference.

### FIELD OF THE INVENTION

The present invention relates to compositions comprising two different polyester polymers and to their use, in particular in cosmetics such as lipsticks. Novel polyesters are also described.

The compositions according to the invention preferably can be applied to substrates, such as the skin of the face or body, lips and keratinous substances, such as the hair, eyelashes, eyebrows and nails.

Additional aspects and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

There exist numerous cosmetic compositions for which properties of gloss of the film deposited, after application to keratinous substances (skin, lips, superficial body growths), are desired. Mention may be made, for example, of lipsticks, nail varnishes or some hair products.

In order to obtain such a result, it is possible to combine specific starting materials, in particular lanolins, with “glossy” oils, such as polybutenes, which, however, exhibit a high viscosity; or esters of fatty acid or alcohol having a high carbon number; or else certain vegetable oils; or also esters resulting from the partial or complete esterification of a hydroxylated aliphatic compound with an aromatic acid, as described in Patent Application EP 1 097 699.

It is also known to combine lanolins with polyesters obtained by sequential reaction of castor oil with isostearic acid and then with succinic acid, as described in U.S. Pat. No. 6,342,527.

In order to improve the gloss of the film deposited, and also its hold, the proposal has also been made to use esters resulting from the condensation of a polyol with a carboxylic acid of “neo” type, in particular in FR 2 838 049.

Mention may also be made of EP 1 457 201, which describes a composition combining a polyester of triglycerides of hydroxylated carboxylic acids and an oil of low molecular weight chosen from polybutylenes, hydrogenated polyisobutylenes, hydrogenated or nonhydrogenated polydecenes, vinylpyrrolidone copolymers, linear fatty acid esters, hydroxylated esters, C₂₄-C₂₈ branched fatty alcohol or fatty acid esters, silicone oils and/or oils of vegetable origin. A description is given, in Patent Application EP 0 792 637, of a composition combining an aromatic ester and a polymer of polybutene or polyisobutene type.

A description is given, in Patent Application EP 1 155 687, of a process which consists in incorporating, in an oily phase composed of a cosmetically acceptable oil, an organopolysiloxane having at least 2 groups capable of establishing hydrogen bonds.

However, these compositions and combinations, even if they significantly improve the gloss, are still considered inadequate from the viewpoint of the long-lasting hold of this gloss over time.

### DESCRIPTION OF THE PREFERRED EMBODIMENTS

The polymers used in the context of the present invention are preferably alkyd resins, which constitute a specific class of polyesters, being the reaction product of polyols and polycarboxylic acids, generally modified by unsaturated fatty acids, such as oleic acid, or by unsaturated oils, for example soybean oil or castor oil.

Cosmetic compositions comprising polyesters have been described in the prior art. Mention may in particular be made of the document FR 2 562 793, which describes the use of sucrose benzoate in combination with toluenesulphonamide/formaldehyde resins; or the document JP61246113, which describes the use of sucrose benzoate in combination with an alkyd resin modified with glycidyl versatate ester. Mention may also be made of WO2002243676, which describes the use of a neopentyl glycol trimellitate adipate polyester resin in combination with alkyl acrylate and methacrylate copolymers. JP58023614 is also known, which describes the use of a modified polyester obtained by condensation of pentaerythritol with cis-4-cyclohexene-1,2-dicarboxylic acid and castor oil fatty acids and then reaction with a dioxirane compound of epoxy resin type; or JP54011244 is also known, which describes the use of a modified polyester obtained by condensation of dipentaerythritol with cyclohexane-1,2-dicarboxylic acid and castor oil fatty acids and then reaction with a dioxirane compound of epoxy resin type.

The polyesters used in the context of the present invention have a different structure from known polyesters. In addition, when they are formulated in combination with specific ingredients, they make it possible to obtain cosmetic properties which are the same as or indeed even better than the performances already obtained with known polyesters.

One aim of the present invention is to provide cosmetic compositions, the gloss of which is improved in comparison with the compositions of the prior art comprising other polyesters, and the hold of the gloss of which is improved over time

The inventor has discovered, surprisingly and unexpectedly, that two specific polyesters result in compositions, the hold of the gloss of which is improved. Compositions, preferably cosmetic compositions, comprising these polyesters make up a part of the invention.

One subject-matter of the present invention is thus a composition, comprising:
at least one polyester capable of being obtained by reaction:
   of at least one polyol comprising 3 to 6 hydroxyl groups;
   of at least one nonaromatic branched monocarboxylic acid;
   of at least one aromatic monocarboxylic acid, and
   of at least one polycarboxylic acid comprising at least 2 carboxyl groups COOH and/or one cyclic anhydride of such a polycarboxylic acid,
at least one polyester capable of being obtained by reaction:
   of at least one polyol comprising 3 to 6 hydroxyl groups;
   of at least one nonaromatic linear monocarboxylic acid;
   of at least one aromatic monocarboxylic acid, and
   of at least one polycarboxylic acid comprising at least 2 carboxyl groups COOH and/or one cyclic anhydride of such a polycarboxylic acid.

Naturally, the two polyesters are different from one another.

The composition of the invention can be provided in any form including the form of a paste, solid or more or less viscous cream. It can be an oil-in-water or water-in-oil emulsion or a stiff or soft anhydrous gel. In particular, it is provided in the form cast as a stick or in a dish and more especially in the form of an anhydrous stiff gel, in particular of an anhydrous stick.

According to another of its aspects, a subject-matter of the present invention is a composition, comprising:
a first benzoic acid/isophthalic acid/isostearic acid/pentaerythritol polymer, and
a second benzoic acid/isophthalic acid/stearic acid/pentaerythritol polymer.

The ratio by weight of the first polymer to the second polymer is advantageously between 50/1 and 2/1, for example between 30/1 and 20/1.

The term “hydrocarbon” is understood to mean a radical or a compound formed essentially, indeed even composed, of carbon and hydrogen atoms and optionally of oxygen, nitrogen, sulphur or phosphorus atoms but not comprising silicon or fluorine atoms. It can comprise alcohol, ether, carboxylic acid, amine and/or amide groups. Preferably, the adjective “hydrocarbon” denotes a radical or a compound composed solely of carbon and hydrogen, and oxygen, atoms.

The term “branched” is understood to mean a compound comprising at least one branching. More generally, the number of branchings of a molecule corresponds to the number of side groups comprising at least one carbon atom and branched on the main chain of the molecule, the main chain corresponding to the longest carbon chain of the molecule (see Organic Chemistry, S. H. Pine, 5th Edition, McGraw-Hill, Chapter 3).

### Polyesters (or Polycondensates)

Polyesters (also known subsequently as polycondensates) are advantageously obtained by reaction of a polyol, a polycarboxylic acid, a nonaromatic branched or linear monocarboxylic acid and an aromatic monocarboxylic acid.

The first polymer can be obtained by reaction of a polyol, a polycarboxylic acid, a nonaromatic branched monocarboxylic acid and an aromatic monocarboxylic acid.

The second polymer is advantageously obtained by reaction of a polyol, a polycarboxylic acid, a nonaromatic linear monocarboxylic acid and an aromatic monocarboxylic acid.

According to one embodiment, the content of nonaromatic monocarboxylic acid is between 5 and 80% by weight, preferably between 20 and 70% by weight, for example from 25 to 65% by weight, with respect to the total weight of the polycondensate.

According to another embodiment, the polyesters are advantageously obtained from the reaction of a polyol, a polycarboxylic acid and at least one nonaromatic monocarboxylic acid, the monocarboxylic acid being in a high content.

The polycondensates are capable of being obtained by esterification/polycondensation, according to methods known to a person skilled in the art, of the constituents described below.

One of the constituents useful in the preparation of the polycondensates according to the invention is a polyol, preferably comprising 3 to 6 hydroxyl groups, in particular 3 or 4 hydroxyl groups. Use may very clearly be made of a mixture of such polyols.

The polyol can in particular be a saturated or unsaturated and linear, branched and/or cyclic carbon, in particular hydrocarbon, compound which comprises 3 to 18 carbon atoms, in particular 3 to 12 carbon atoms, indeed even 4 to 10 carbon atoms, and 3 to 6 hydroxyl (OH) groups and which can additionally comprise one or more oxygen atoms intercalated in the chain (ether functional group).

The polyol is preferably a saturated, linear or branched, hydrocarbon compound comprising 3 to 18 carbon atoms, in particular 3 to 12 carbon atoms, indeed even 4 to 10 carbon atoms, and 3 to 6 hydroxyl (OH) groups.

It can be chosen, alone or as a mixture, from:
triols, such as 1,2,4-butanetriol, 1,2,6-hexanetriol, trimethylolethane, trimethylolpropane or glycerol;
tetraols, such as pentaerythritol (tetramethylol-methane), erythritol, diglycerol or ditrimethylolpropane;
pentols, such as xylitol,
hexyls, such as sorbitol and mannitol; or also dipentaerythritol or triglycerol.

Preferably, the polyol is chosen from glycerol, pentaerythritol, diglycerol, sorbitol and their mixtures; and better still the polyol is a tetraol, such as pentaerythritol.

The polyol, or the polyol mixture, preferably represents 10 to 30% by weight, in particular 12 to 25% by weight and better still 14 to 22% by weight of the total weight of the final polycondensate.

Another constituent for the preparation of the first polyester according to the invention is a nonaromatic branched monocarboxylic acid. The nonaromatic branched monocarboxylic acid can be saturated or unsaturated, comprising 6 to 32 carbon atoms, in particular 8 to 28 carbon atoms and better still 10 to 24, indeed even 12 to 20, carbon atoms. Use may very obviously be made of a mixture of such nonaromatic monocarboxylic acids. The term “nonaromatic branched monocarboxylic acid” is understood to mean a compound of formula RCOOH in which R is a saturated or unsaturated and branched hydrocarbon radical comprising 5 to 31 carbon atoms, in particular 7 to 27 carbon atoms and better still 9 to 23 carbon atoms, indeed even 11 to 19 carbon atoms. Preferably, the R radical is saturated. Better still, the R radical is a branched C₅-C₃₁, indeed even C₁₁-C₂₁, radical.

In a specific embodiment of the invention, the non-aromatic branched monocarboxylic acid exhibits a melting point of greater than or equal to 25° C., in particular of greater than or equal to 28° C., indeed even 30° C.; this is because it has been found that, when such an acid is employed, in particular in a large amount, it is possible, on the one hand, to obtain good gloss and good hold of the gloss and, on the other hand, to reduce the amount of waxes normally present in the composition envisaged.

Mention may be made, among nonaromatic branched monocarboxylic acids capable of being employed, of, alone or as a mixture:

isoheptanoic acid, 4-ethylpentanoic acid, 2-ethylhexanoic acid, 4,5-dimethylhexanoic acid, 2-heptylheptanoic acid, 3,5,5-trimethylhexanoic acid, isooctanoic acid, isononanoic acid or isostearic acid.

Preferably, use may be made of 2-ethylhexanoic acid, isooctanoic acid, isoheptanoic acid, isononanoic acid, isostearic acid and their mixtures and better still isostearic acid.

The nonaromatic branched monocarboxylic acid or the mixture of the acids preferably represents 30 to 80% by weight, in particular 40 to 75% by weight, indeed even 45 to 70% by weight and better still 50 to 65% by weight of the total weight of the final polycondensate.

Another constituent for the preparation of the second polyester according to the invention is a nonaromatic linear monocarboxylic acid. The nonaromatic monocarboxylic acid can be saturated or unsaturated, comprising 6 to 32 carbon atoms, in particular 8 to 28 carbon atoms and better still 10 to 24, indeed even 12 to 20, carbon atoms. It is very obviously possible to use a mixture of such nonaromatic monocarboxylic acids. The term “nonaromatic monocarboxylic acid” is understood to mean a compound of formula RCOOH in which R is a saturated or unsaturated, linear, hydrocarbon radical comprising 5 to 31 carbon atoms, in particular 7 to 27 carbon atoms and better still 9 to 23 carbon atoms, indeed even 11 to 19 carbon atoms.

Preferably, the R radical is saturated. Better still, the R radical is linear or branched and preferably a C₅-C₃₁, indeed even C₁₁-C₂₁, radical.

In a specific embodiment of the invention, the nonaromatic monocarboxylic acid exhibits a melting point of greater than or equal to 25° C., in particular of greater than or equal to 28° C., indeed even 30° C.; this is because it has been found that, when such an acid is employed, in particular in a large amount, it is possible, on the one hand, to obtain good gloss and the hold of the gloss and, on the other hand, to reduce the amount of waxes normally present in the composition envisaged.

Mention may be made, among nonaromatic linear monocarboxylic acids capable of being employed, of, alone or as a mixture:
the saturated monocarboxylic acids, such as caproic acid, caprylic acid, octanoic acid, nonanoic acid, decanoic acid, lauric acid, tridecanoic acid, myristic acid, palmitic acid, stearic acid, arachidic acid, behenic acid, cerotic (hexacosanoic) acid, cyclopentanecarboxylic acid, cyclopentaneacetic acid, 3-cyclopentylpropionic acid, cyclohexanecarboxylic acid, cyclohexylacetic acid or 4-cyclohexylbutyric acid;
unsaturated but nonaromatic monocarboxylic acids, such as caproleic acid, obtusilic acid, undecylenic acid, dodecylenic acid, linderic acid, myristoleic acid, physeteric acid, tsuzuic acid, palmitoleic acid, oleic acid, petroselinic acid, vaccenic acid, elaidic acid, gondoic acid, gadoleic acid, erucic acid, cetoleic acid, nervonic acid, linoleic acid, linolenic acid or arachidonic acid.

Mention may be made, among the nonaromatic linear monocarboxylic acids mentioned above having a melting point of greater than or equal to 25° C., of, alone or as a mixture:
among saturated monocarboxylic acids: decanoic (capric) acid, lauric acid, tridecanoic acid, myristic acid, palmitic acid, stearic acid, arachidic acid, behenic acid or cerotic (hexacosanoic) acid;
among unsaturated but nonaromatic monocarboxylic acids: petroselinic acid, vaccenic acid, elaidic acid, gondoic acid, gadoleic acid, erucic acid or nervonic acid.

Preferably, use may be made of lauric acid, myristic acid, nonanoic acid, palmitic acid, stearic acid, behenic acid and their mixtures and better still stearic acid alone.

The nonaromatic linear monocarboxylic acid or the mixture of the acids preferably represents 30 to 80% by weight, in particular 40 to 75% by weight, indeed even 45 to 70% by weight and better still 50 to 65% by weight of the total weight of the final polycondensate.

Another preferred constituent for the preparation of the polycondensates according to the invention is an aromatic monocarboxylic acid. This acid can comprise 7 to 11 carbon atoms and is in addition optionally substituted by 1 to 3 saturated or unsaturated and linear, branched and/or cyclic alkyl radicals which comprise 1 to 32 carbon atoms, in particular 2 to 12, indeed even 3 to 8, carbon atoms. It is possible to use a mixture of such aromatic monocarboxylic acids.

The term “aromatic monocarboxylic acid” is understood to mean a compound of formula R′COOH in which R′ is an aromatic hydrocarbon radical comprising 6 to 10 carbon atoms, and in particular the benzoic and naphthoic radicals.

The R′ radical can additionally be substituted by 1 to 3 saturated or unsaturated and linear, branched and/or cyclic alkyl radicals which comprise 1 to 32 carbon atoms, in particular 2 to 12, indeed even 3 to 8, carbon atoms, and which in particular are chosen from methyl, ethyl, propyl, isopropyl, butyl, isobutyl, tert-butyl, pentyl, isopentyl, neopentyl, cyclopentyl, hexyl, cyclohexyl, heptyl, isoheptyl, octyl or isooctyl.

Mention may be made, among aromatic monocarboxylic acids capable of being employed, of, alone or as a mixture, benzoic acid, o-toluic acid, m-toluic acid, p-toluic acid, 1-naphthoic acid, 2-naphthoic acid, 4-(tert-butyl)benzoic acid, 1-methyl-2-naphthoic acid or 2-isopropyl-1-naphthoic acid.

Use is preferably made of benzoic acid, 4-(tert-butyl)-benzoic acid, o-toluic acid, m-toluic acid or 1-naphthoic acid, alone or as mixtures, and better still benzoic acid alone.

The aromatic monocarboxylic acid or the mixture of the acids preferably represents 0.1 to 10% by weight, in particular 0.5 to 9.95% by weight, better still from 1 to 9.5% by weight, indeed even 1.5 to 8% by weight, of the total weight of the final polycondensate.

The polyester can be obtained from a saturated or unsaturated nonaromatic branched monocarboxylic acid which comprises 10 to 32 carbon atoms, in particular 12 to 28 carbon atoms and better still 12 to 24 carbon atoms and which has a melting point of greater than or equal to 25° C., in particular of greater than or equal to 28° C., indeed even 30° C. It is very obviously possible to use a mixture of such nonaromatic monocarboxylic acids.

It has been found that, when such an acid is employed in the amounts indicated, it is possible, on the one hand, to obtain good gloss and the hold of the gloss and, on the other hand, to reduce the amount of waxes normally present in the composition envisaged.

The term “nonaromatic branched monocarboxylic acid” is understood to mean a compound of formula RCOOH in which R is a saturated or unsaturated hydrocarbon radical comprising 9 to 31 carbon atoms, in particular 11 to 27 carbon atoms and better still 11 to 23 carbon atoms. Preferably, the R radical is saturated. Better still, the R radical is linear or branched and preferably a C₁₁-C₂₁ radical.

The nonaromatic branched monocarboxylic acid with a melting point of greater than or equal to 25° C. or the mixture of the acids preferably represents 22 to 80% by weight, in particular 25 to 75% by weight, indeed even 27 to 70% by weight and better still 28 to 65% by weight of the total weight of the final polycondensate.

The polyester can be obtained from a saturated or unsaturated nonaromatic branched monocarboxylic acid which comprises 6 to 32 carbon atoms, in particular 8 to 28 carbon atoms and better still 10 to 20, indeed even 12 to 18, carbon atoms and which can have a melting point of strictly less than 25° C., in particular less than 20° C., indeed even 15° C. It is very obviously possible to use a mixture of such nonaromatic monocarboxylic acids.

The term “nonaromatic branched monocarboxylic acid” is understood to mean a compound of formula RCOOH in which R is a saturated or unsaturated and linear, branched and/or cyclic hydrocarbon radical comprising 5 to 31 carbon atoms, in particular 7 to 27 carbon atoms and better still 9 to 19 carbon atoms, indeed even 11 to 17 carbon atoms.

Preferably, the R radical is saturated. Better still, the R radical is linear or branched and preferably a C₅-C₃₁ radical. Mention may be made, among nonaromatic monocarboxylic acids having a melting point of less than 25° C. which are capable of being employed, of, alone or as a mixture:
among saturated monocarboxylic acids: isoheptanoic acid, 4-ethylpentanoic acid, 2-ethylhexanoic acid, 4,5-dimethylhexanoic acid, 2-heptylheptanoic acid, 3,5,5-trimethylhexanoic acid, isooctanoic acid, isononanoic acid or isostearic acid.

Preferably, use may be made of isooctanoic acid, isononanoic acid, isostearic acid and their mixtures and better still isostearic acid alone.

The nonaromatic branched monocarboxylic acid with a melting point of less than 25° C. or the mixture of the acids preferably represents 0.1 to 35% by weight, in particular 0.5 to 32% by weight, indeed even 1 to 30% by weight and better still 2 to 28% by weight of the total weight of the final polycondensate.

Another constituent for the preparation of the polycondensates according to the invention is a saturated or unsaturated, indeed even aromatic, and linear, branched and/or cyclic polycarboxylic acid comprising at least 2 carboxyl COOH groups, in particular 2 to 4 COOH groups, and/or a cyclic anhydride of such a polycarboxylic acid. It is very obviously possible to use a mixture of such polycarboxylic acids and/or anhydrides.

The polycarboxylic acid can in particular be chosen from saturated or unsaturated, indeed even aromatic, and linear, branched and/or cyclic polycarboxylic acids comprising 3 to 50, in particular 3 to 40, carbon atoms, especially 3 to 36, indeed even 3 to 18 and better still 4 to 12 carbon atoms, indeed even 4 to 10 carbon atoms.

The acid comprises at least two carboxyl COOH groups, preferably from 2 to 4 COOH groups.

Preferably, the polycarboxylic acid is aliphatic and comprises 3 to 36 carbon atoms, in particular 3 to 18 carbon atoms, indeed even 4 to 12 carbon atoms, or else the polycarboxylic acid is aromatic and comprises 8 to 12 carbon atoms. It preferably comprises 2 to 4 COOH groups.

The cyclic anhydride of such a polycarboxylic acid can in particular correspond to one of the following formulae:

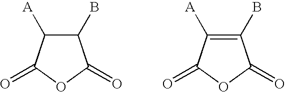

in which the A and B groups are, independently of one another:
a hydrogen atom,
a saturated or unsaturated and linear, branched and/or cyclic aliphatic carbon radical or else an aromatic carbon radical comprising 1 to 16 carbon atoms, in particular 2 to 10 carbon atoms, indeed even 4 to 8 carbon atoms, in particular methyl or ethyl;
or else A and B, taken together, form a saturated or unsaturated, indeed even aromatic, ring comprising a total of 5 to 7, in particular 6, carbon atoms.

Preferably, A and B represent a hydrogen atom or together form an aromatic ring comprising a total of 6 carbon atoms.

Mention may be made, among polycarboxylic acids or their anhydrides capable of being employed, of, alone or as a mixture:
dicarboxylic acids, such as decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, tetrahydrophthalic acid, hexahydrophthalic acid, pimelic acid, sebacic acid, azelaic acid, glutaric acid, adipic acid, fumaric acid, maleic acid, itaconic acid or fatty acid dimers (in particular C₃₆ dimers), such as the products sold under the names Pripol 1006, 1009, 1013 and 1017 by Uniqema;
tricarboxylic acids, such as cyclohexanetricarboxylic acid, trimellitic acid, 1,2,3-benzenetricarboxylic acid or 1,3,5-benzenetricarboxylic acid;
tetracarboxylic acids, such as butanetetracarboxylic acid and pyromellitic acid;
cyclic anhydrides of these acids and in particular phthalic anhydride, trimellitic anhydride, maleic anhydride and succinic anhydride.

Preferably, use may be made of adipic acid, phthalic anhydride and/or isophthalic acid and better still isophthalic acid alone.

The polycarboxylic acid and/or its cyclic anhydride preferably represents 5 to 40% by weight, in particular 10 to 30% by weight and better still 14 to 25% by weight of the total weight of the final polycondensate.

The polycondensate can additionally comprise a silicone having a hydroxyl (OH) and/or carboxyl (COOH) functional group. It can comprise 1 to 3 hydroxyl and/or carboxyl functional groups and preferably comprises two hydroxyl functional groups or else two carboxyl functional groups. These functional groups can be situated at the chain end or in the chain but advantageously at the chain end.

Use is preferably made of silicones having a weight-average molecular weight (Mw) of between 300 and 20 000, in particular 400 and 10 000, indeed even 800 and 4000.

This silicone can be of formula:

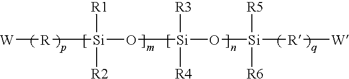

in which:
W and W′ are, independently of one another, OH or COOH; preferably, W═W′;
p and q are, independently of one another, equal to 0 or 1,
R and R′ are, independently of one another, a divalent carbon, in particular hydrocarbon, radical which is saturated or unsaturated, indeed even aromatic, and linear, branched and/or cyclic, which comprises 1 to 12 carbon atoms, in particular 2 to 8 carbon atoms, and which optionally comprises, in addition, 1 or more heteroatoms chosen from O, S and N, in particular C (ether);
   in particular, R and/or R′ can be of formula —(CH₂)ₐ— with a=1-12 and in particular methylene, ethylene, propylene or phenylene;
   or else of formula —[(CH₂)ₓO]_{z}— with x=1, 2 or 3 and z=1-10; in particular, x=2 or 3 and z=1-4; and better still x=3 and z=1;
R1 to R6 are, independently of one another, a saturated or unsaturated, indeed even aromatic, linear, branched and/or cyclic carbon radical comprising 1 to 20 carbon atoms, in particular 2 to 12 carbon atoms; preferably, R1 to R6 are saturated or else aromatic and can in particular be chosen from alkyl radicals, in particular methyl, ethyl, propyl, isopropyl, butyl, pentyl, hexyl, octyl, decyl, dodecyl and octadecyl radicals, cycloalkyl radicals, in particular the cyclohexyl radical, aryl radicals, in particular phenyl and naphthyl radicals, arylalkyl radicals, in particular benzyl and phenylethyl radicals, and also the tolyl and xylyl radicals;
m and n are, independently of one another, integers between 1 and 140 and are such that the weight-average molecular weight (Mw) of the silicone is between 300 and 20 000, in particular between 400 and 10 000, indeed even between 800 and 4000.

Mention may in particular be made of α,ω-dihydroxy- or α,ω-dicarboxypolyalkylsiloxanes and in particular α,ω-dihydroxypolydimethylsiloxanes and α,ω-dicarboxy-polydimethylsiloxanes; α,ω-dihydroxy- or α,ω-dicarboxy-polyarylsiloxanes and in particular α,ω-dihydroxy- or α,ω-dicarboxypolyphenylsiloxanes; polyarylsiloxanes having silanol functional groups, such as polyphenylsiloxane; polyalkylsiloxanes having silanol functional groups, such as polydimethylsiloxane; or polyaryl/alkylsiloxanes having silanol functional groups, such as polyphenyl/methylsiloxane or polyphenyl/propylsiloxane.

Use will very particularly be made of α,ω-dihydroxy-polydimethylsiloxanes with a weight-average molecular weight (Mw) of between 400 and 10 000, indeed even between 500 and 5000 and in particular between 800 and 4000.

When it is present, the silicone can preferably represent 0.1 to 15% by weight, in particular 1 to 10% by weight, indeed even 2 to 8% by weight, of the weight of the polycondensate.

According to one embodiment, the first polyester is capable of being obtained, or is obtained, by reaction:
of at least one polyol comprising 3 to 6 hydroxyl groups;
of at least one nonaromatic branched monocarboxylic acid comprising 6 to 32 carbon atoms;
of at least one aromatic monocarboxylic acid comprising 7 to 11 carbon atoms;
of at least one polycarboxylic acid comprising at least 2 carboxyl COOH groups and/or one cyclic anhydride of such a polycarboxylic acid.

According to one embodiment, the second polyester is capable of being obtained, or is obtained, by reaction:
of at least one polyol comprising 3 to 6 hydroxyl groups;
of at least one nonaromatic linear monocarboxylic acid comprising 6 to 32 carbon atoms;
of at least one aromatic monocarboxylic acid comprising 7 to 11 carbon atoms;
of at least one polycarboxylic acid comprising at least 2 carboxyl COOH groups and/or one cyclic anhydride of such a polycarboxylic acid.

Preferably, the nonaromatic monocarboxylic acid does not comprise a free OH group.

According to one embodiment, the polycondensate can be obtained by reaction:
of 10 to 30% by weight, with respect to the total weight of the polycondensate, of at least one polyol comprising 3 to 6 hydroxyl groups;
of 30 to 80% by weight, with respect to the total weight of the polycondensate, of at least one saturated or unsaturated (linear or branched, according to the polyester which it is desired to prepare) nonaromatic monocarboxylic acid comprising 6 to 32 carbon atoms;
of 0.1 to 10% by weight, with respect to the total weight of the polycondensate, of at least one aromatic monocarboxylic acid comprising 7 to 11 carbon atoms, optionally in addition substituted by 1 to 3 saturated or unsaturated and linear, branched and/or cyclic alkyl radicals which comprise 1 to 32 carbon atoms;
of 5 to 40% by weight, with respect to the total weight of the polycondensate, of at least one saturated or unsaturated, indeed even aromatic, and linear, branched and/or cyclic polycarboxylic acid comprising at least 2 carboxyl COOH groups, in particular 2 to 4 COOH groups, and/or one cyclic anhydride of such a polycarboxylic acid.

According to one embodiment, the polycondensate is capable of being obtained, or is obtained, by reaction:
of 15 to 30% by weight, with respect to the total weight of the polycondensate, of at least one polyol comprising 3 to 6 hydroxyl groups;
of 5 to 40% by weight, with respect to the total weight of the polycondensate, of at least one saturated or unsaturated (linear or branched, according to the polyester which it is desired to prepare) nonaromatic monocarboxylic acid comprising 6 to 32 carbon atoms;
of 10 to 55% by weight, with respect to the total weight of the polycondensate, of at least one aromatic monocarboxylic acid comprising 7 to 11 carbon atoms, optionally in addition substituted by 1 to 3 saturated or unsaturated and linear, branched and/or cyclic alkyl radicals which comprise 1 to 32 carbon atoms;
of 10 to 25% by weight, with respect to the total weight of the polycondensate, of at least one saturated or unsaturated, indeed even aromatic, and linear, branched and/or cyclic polycarboxylic acid comprising at least 2 carboxyl COOH groups, in particular 2 to 4 COOH groups, and/or one cyclic anhydride of such a polycarboxylic acid.

Preferably, the first polyester is capable of being obtained, or is obtained, by reaction:
of at least one polyol chosen, alone or as a mixture, from 1,2,6-hexanetriol, trimethylolethane, trimethylolpropane, glycerol, pentaerythritol, erythritol, diglycerol, ditrimethylolpropane, xylitol, sorbitol, mannitol, dipentaerythritol and/or triglycerol;
preferably present in an amount of 10 to 30% by weight, in particular 12 to 25% by weight and better still 14 to 22% by weight, with respect to the total weight of the final polycondensate;
of at least one nonaromatic branched monocarboxylic acid chosen, alone or as a mixture from isoheptanoic acid, 4-ethylpentanoic acid, 2-ethylhexanoic acid, 4,5-dimethylhexanoic acid, 2-heptylheptanoic acid, 3,5,5-trimethylhexanoic acid, isooctanoic acid, isononanoic acid or isostearic acid;
preferably present in an amount of 30 to 80% by weight, in particular 40 to 75% by weight and better still 45 to 70% by weight, with respect to the total weight of the final polycondensate;
of at least one aromatic monocarboxylic acid chosen, alone or as a mixture, from benzoic acid, o-toluic acid, m-toluic acid, p-toluic acid, 1-naphthoic acid, 2-naphthoic acid, 4-(tert-butyl)benzoic acid, 1-methyl-2-naphthoic acid or 2-isopropyl-1-naphthoic acid;
preferably present in an amount of 0.1 to 10% by weight, in particular 1 to 9.5% by weight, indeed even 1.5 to 8% by weight, with respect to the total weight of the final polycondensate; and
of at least one polycarboxylic acid or one of its anhydrides chosen, alone or as a mixture, from decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid, maleic acid, cyclohexanetricarboxylic acid, trimellitic acid, 1,2,3-benzenetricarboxylic acid, 1,3,5-benzenetricarboxylic acid, butanetetracarboxylic acid, pyromellitic acid, phthalic anhydride, trimellitic anhydride, maleic anhydride and succinic anhydride;
preferably present in an amount of 5 to 40% by weight, in particular 10 to 30% by weight and better still 14 to 25% by weight, with respect to the total weight of the final polycondensate.

Preferably, the second polyester is capable of being obtained, or is obtained, by reaction:
of at least one polyol chosen, alone or as a mixture, from 1,2,6-hexanetriol, trimethylolethane, trimethylolpropane, glycerol, pentaerythritol, erythritol, diglycerol, ditrimethylolpropane, xylitol, sorbitol, mannitol, dipentaerythritol and/or triglycerol;
preferably present in an amount of 10 to 30% by weight, in particular 12 to 25% by weight and better still 14 to 22% by weight, with respect to the total weight of the final polycondensate;
of at least one nonaromatic linear monocarboxylic acid chosen, alone or as a mixture, from caproic acid, caprylic acid, octanoic acid, nonanoic acid, decanoic acid, lauric acid, tridecanoic acid, myristic acid, palmitic acid, stearic acid, arachidic acid, behenic acid or cerotic (hexacosanoic) acid;
preferably present in an amount of 30 to 80% by weight, in particular 40 to 75% by weight and better still 45 to 70% by weight, with respect to the total weight of the final polycondensate;
of at least one aromatic monocarboxylic acid chosen, alone or as a mixture, from benzoic acid, o-toluic acid, m-toluic acid, p-toluic acid, 1-naphthoic acid, 2-naphthoic acid, 4-(tert-butyl)benzoic acid, 1-methyl-2-naphthoic acid or 2-isopropyl-1-naphthoic acid;
preferably present in an amount of 0.1 to 10% by weight, in particular 1 to 9.5% by weight, indeed even 1.5 to 8% by weight, with respect to the total weight of the final polycondensate; and
of at least one polycarboxylic acid or one of its anhydrides chosen, alone or as a mixture, from decanedioic acid, dodecanedioic acid, cyclopropanedicarboxylic acid, cyclohexanedicarboxylic acid, cyclobutanedicarboxylic acid, naphthalene-1,4-dicarboxylic acid, naphthalene-2,3-dicarboxylic acid, naphthalene-2,6-dicarboxylic acid, suberic acid, oxalic acid, malonic acid, succinic acid, phthalic acid, terephthalic acid, isophthalic acid, pimelic acid, sebacic acid, azelaic acid, glutaric acid, adipic acid, fumaric acid, maleic acid, cyclohexanetricarboxylic acid, trimellitic acid, 1,2,3-benzenetricarboxylic acid, 1,3,5-benzenetricarboxylic acid, butanetetracarboxylic acid, pyromellitic acid, phthalic anhydride, trimellitic anhydride, maleic anhydride and succinic anhydride;
preferably present in an amount of 5 to 40% by weight, in particular 10 to 30% by weight and better still 14 to 25% by weight, with respect to the total weight of the final polycondensate.

According to another embodiment, the first polycondensate and the second polycondensate are both capable of being obtained by reaction:
of 10 to 30% by weight, with respect to the total weight of the polycondensate, of at least one polyol comprising 3 to 6 hydroxyl groups;
of 22 to 80% by weight, with respect to the total weight of the polycondensate, of at least one saturated or unsaturated nonaromatic branched monocarboxylic acid comprising 10 to 32 carbon atoms and having a melting point of greater than or equal to 25° C.;
of 0.1 to 35% by weight, with respect to the total weight of the polycondensate, of at least one saturated or unsaturated nonaromatic linear monocarboxylic acid comprising 6 to 32 carbon atoms and having a melting point of strictly less than 25° C.;
of 0.1 to 10% by weight, with respect to the total weight of the polycondensate, of at least one aromatic monocarboxylic acid comprising 7 to 11 carbon atoms, optionally in addition substituted by 1 to 3 saturated or unsaturated and linear, branched and/or cyclic alkyl radicals which comprise 1 to 32 carbon atoms;
of 5 to 40% by weight, with respect to the total weight of the polycondensate, of at least one saturated or unsaturated, indeed even aromatic, and linear, branched and/or cyclic polycarboxylic acid comprising at least 2 carboxyl COOH groups, in particular 2 to 4 COOH groups, and/or one cyclic anhydride of such a polycarboxylic acid.

Preferably, the first polycondensate is capable of being obtained, or is obtained, by reaction:
of at least one polyol chosen, alone or as a mixture, from glycerol, pentaerythritol, sorbitol and their mixtures and better still pentaerythritol alone; present in an amount of 10 to 30% by weight, in particular 12 to 25% by weight and better still 14 to 22% by weight, with respect to the total weight of the final polycondensate;
of at least one nonaromatic branched monocarboxylic acid chosen, alone or as a mixture, from 2-ethylhexanoic acid, isooctanoic acid, lauric acid, palmitic acid, isostearic acid, isononanoic acid, stearic acid, behenic acid and their mixtures and better still isostearic acid alone or stearic acid alone;
present in an amount of 30 to 80% by weight, in particular 40 to 75% by weight and better still 45 to 70% by weight, with respect to the total weight of the final polycondensate;
of at least one aromatic monocarboxylic acid chosen, alone or as a mixture, from benzoic acid, o-toluic acid, m-toluic acid or 1-naphthoic acid and better still benzoic acid alone; present in an amount of 0.1 to 10% by weight, in particular 1 to 9.5% by weight, indeed even 1.5 to 8% by weight, with respect to the total weight of the final polycondensate; and
of at least one polycarboxylic acid or one of its anhydrides chosen, alone or as a mixture, from phthalic anhydride and isophthalic acid and better still isophthalic acid alone; present in an amount of 5 to 40% by weight, in particular 10 to 30% by weight and better still 14 to 25% by weight, with respect to the total weight of the final polycondensate.

Preferably, the first polycondensate and/or the second polycondensate exhibits:
an acid number, expressed as mg of potassium hydroxide per g of polycondensate, of greater than or equal to 1, in particular of between 2 and 30 and better still of between 2.5 and 15; and/or
a hydroxyl number, expressed as mg of potassium hydroxide per g of polycondensate, of greater than or equal to 40, in particular of between 40 and 120 and better still of between 45 and 80.

These acid and hydroxyl numbers can be easily determined by a person skilled in the art by the usual analytical methods.

Preferably, the first polycondensate and/or the second polycondensate exhibits a weight-average molecular weight (Mw) of between 1500 and 300 000, indeed even between 2000 and 200 000 and in particular between 3000 and 100 000.

The average molecular weight can be determined by gel permeation chromatography or by light scattering, according to the solubility of the polymer under consideration.

Preferably, the first polycondensate and/or the second polycondensate exhibits a viscosity, measured at 110° C., of between 20 and 4000 mPa·s, in particular between 30 and 3500 mPa·s, indeed even between 40 and 3000 mPa·s and better still between 50 and 2500 mPa·s. This viscosity is measured in the way described before the examples.

The first polycondensate and/or the second polycondensate can be prepared by the esterification/polycondensation processes conventionally employed by a person skilled in the art. By way of illustration, a general preparation process comprises:
in mixing the polyol and the aromatic and nonaromatic monocarboxylic acids,
in heating the mixture under an inert atmosphere, first up to the melting point (generally 100-130° C.) and subsequently to a temperature of between 150 and 220° C. until the monocarboxylic acids have been completely consumed (reached when the acid number is less than or equal to 1), preferably while distilling off, as it is formed, the water formed, then
in optionally cooling the mixture to a temperature of between 90 and 150° C.,
in adding the polycarboxylic acid and/or the cyclic anhydride and optionally the silicone having hydroxyl or carboxyl functional groups, all at once or sequentially, then
in again heating to a temperature of less than or equal to 220° C., in particular of between 170 and 220° C., preferably while continuing to remove the water formed, until the characteristics required in terms of acid number, viscosity, hydroxyl number and solubility are obtained.

It is possible to add conventional esterification catalysts, for example of sulphonic acid type (in particular at a concentration by weight of between 1 and 10%) or titanate type (in particular at a concentration by weight of between 5 and 100 ppm).

It is also possible to carry out the reaction, in whole or part, in an inert solvent, such as xylene, and/or under a reduced pressure, in order to facilitate the removal of the water.

Advantageously, neither catalyst nor solvent is used.

The preparation process can additionally comprise a stage of addition of at least one antioxidant to the reaction medium, in particular at a concentration by weight of between 0.01 and 1%, with respect to the total weight of monomers, so as to limit possible decomposition events related to prolonged heating.

The antioxidant can be of primary type or of secondary type and can be chosen from hindered phenols, aromatic secondary amines, organophosphorus compounds, sulphur compounds, lactones, bisphenol acrylates and their mixtures.

Mention may in particular be made, among particularly preferred antioxidants, of BHT, BHA, TBHQ, 1,3,5-trimethyl-2,4,6-tris(3,5-di(tert-butyl)-4-hydroxybenzyl)-benzene, octadecyl 3,5-di(tert-butyl)-4-hydroxy-cinnamate, tetrakis-methylene-3-(3,5-di(tert-butyl)-4-hydroxyphenyl)propionate methane, octadecyl 3-(3,5-di(tert-butyl)-4-hydroxyphenyl)propionate, 2,5-di(tert-butyl)hydroquinone, 2,2-methylenebis(4-methyl-6-(tert-butyl)phenol), 2,2-methylenebis(4-ethyl-6-(tert-butyl)-phenol), 4,4-butylidenebis(6-(tert-butyl)-m-cresol), N,N′-hexamethylene bis(3,5-di(tert-butyl)-4-hydroxy-hydrocinnamamide), pentaerythritol tetrakis(3-(3,5-di(tert-butyl)-4-hydroxyphenyl)propionate), in particular that sold by CIBA under the name Irganox 1010, octadecyl 3-(3,5-di(tert-butyl)-4-hydroxyphenyl)-propionate, in particular that sold by CIBA under the name Irganox 1076, 1,3,5-tris(3,5-di(tert-butyl)-4-hydroxybenzyl)-1,3,5-triazine-2,4,6(1H,3H,5H)-trione, in particular that sold by Mayzo of Norcross, Ga., under the name BNX 3114, distearyl pentaerythritol diphosphite, tris(2,4-di(tert-butyl)phenyl)phosphite, in particular that sold by CIBA under the name Irgafos 168, dilauryl thiodipropionate, in particular that sold by CIBA under the name Irganox PS800, bis(2,4-di(tert-butyl)) pentaerythritol diphosphite, in particular that sold by CIBA under the name Irgafos 126, bis(2,4-bis[2-phenylpropan-2-yl]phenyl) pentaerythritol diphosphite, triphenyl phosphite, 2,4-di(tert-butyl)phenyl pentaerythritol diphosphite, in particular that sold by GE Specialty Chemicals under the name Ultranox 626, tris(nonylphenyl) phosphite, in particular that sold by CIBA under the name Irgafos TNPP, the 1:1 mixture of N,N′-hexamethylene bis(3,5-di(tert-butyl)-4-hydroxy-hydrocinnamamide) and of tris(2,4-di(tert-butyl)phenyl) phosphite, in particular that sold by CIBA under the name Irganox B 1171, tris(2,4-di(tert-butyl)phenyl) phosphite, in particular that sold by CIBA under the name Irgafos P-EPQ, distearyl thiodipropionate, in particular that sold by CIBA under the name Irganox PS802, 2,4-bis(octylthiomethyl)-o-cresol, in particular that sold by CIBA under the name Irganox 1520, or 4,6-bis(dodecylthiomethyl)-o-cresol, in particular that sold by CIBA under the name Irganox 1726.

The first polyester can advantageously be present in a total amount of between 1 and 50% by weight, in particular between 10 and 45% by weight, indeed even between 10 and 20% by weight, with respect to the weight of the composition.

The second polyester can advantageously be present in a total amount of between 0.1 and 20% by weight, in particular between 0.2 and 10% by weight, indeed even between 0.5 and 2% by weight, with respect to the weight of the composition.

The total amount of polyesters present in the compositions depends, of course, on the type of composition and properties desired and can vary within a very broad range, generally of between 0.1 and 70% by weight, preferably between 1 and 50% by weight, in particular between 10 and 45% by weight, indeed even between 20 and 40% by weight and better still between 25 and 35% by weight, with respect to the weight of the cosmetic composition.

According to one embodiment, the total amount of polycondensates is between 10 and 20% by weight.

### Nonvolatile Oil

The composition according to the invention advantageously comprises a nonvolatile oil.

The nonvolatile oil can represent, e.g., 1 to 90% by weight of the composition, in particular from 5 to 75% by weight, especially from 10 to 60% by weight, indeed even from 25 to 55% by weight, of the total weight of the composition.

According to one embodiment, the nonvolatile oil can represent from 35 to 60% by weight.

Within the meaning of the present invention, the term “nonvolatile oil” is understood to mean an oil having a vapour pressure of less than 0.13 Pa. The nonvolatile oils can be hydrocarbon oils, silicone oils, fluorinated oils or their mixtures.

Within the meaning of the present invention, the term “silicone oil” is understood to mean an oil comprising at least one silicon atom and in particular at least one Si—O group.

The term “hydrocarbon oil” is understood to mean an oil comprising mainly hydrogen and carbon atoms and optionally oxygen, nitrogen, sulphur and/or phosphorus atoms.

The term “hydrocarbide” is understood to mean an oil comprising only hydrogen and carbon atoms.

The nonvolatile oils can be chosen in particular from nonvolatile hydrocarbon oils, if appropriate fluorinated, and/or nonvolatile silicone oils.

Mention may in particular be made, as nonvolatile hydrocarbon oil, of:
hydrocarbon oils of vegetable origin, such as phytostearyl esters, for example phytostearyl oleate, phytostearyl isostearate and lauroyl/octyldodecyl/phytostearyl glutamate (Ajinomoto, Eldew PS203), triglycerides composed of esters of fatty acids and of glycerol, the fatty acids of which can have varied chain lengths from C₄ to C₂₄, it being possible for these chains to be linear or branched and saturated or unsaturated; these oils are in particular heptanoic or octanoic triglycerides; wheat germ, sunflower, grape seed, sesame, maize, apricot, castor, shea, avocado, olive, soybean, sweet almond, palm, rapeseed, cottonseed, hazelnut, macadamia, jojoba, alfalfa, poppy, pumpkinseed, cucumber, blackcurrant seed, evening primrose, millet, barley, quinoa, rye, safflower, candlenut, passionflower or musk rose oil; shea butter; or triglycerides of caprylic/capric acids, such as those sold by Stéarineries Dubois or those sold under the names Miglyol 810®, 812® and 818® by Dynamit Nobel,
synthetic ethers having from 10 to 40 carbon atoms;
linear or branched hydrocarbides of mineral or synthetic origin, such as liquid petrolatum, polydecenes, hydrogenated polyisobutene, such as Parleam®, squalane and their mixtures, in particular hydrogenated polyisobutene,
synthetic esters, such as oils of formula R₁COOR₂ in which R₁ represents the residue of a linear or branched acid comprising from 1 to 40 carbon atoms, and R₂ represents a hydrocarbon chain, in particular a branched hydrocarbon chain, comprising from 1 to 40 carbon atoms, provided that R₁+R₂≧10.

The esters can in particular be chosen from esters, in particular fatty acid esters, such as, for example: cetearyl octanoate, esters of isopropyl alcohol, such as isopropyl myristate or isopropyl palmitate, ethyl palmitate, 2-ethylhexyl palmitate, isopropyl stearate or isostearate, isostearyl isostearate, octyl stearate, hydroxylated esters, such as isostearyl lactate or octyl hydroxystearate, diisopropyl adipate, heptanoates and in particular isostearyl heptanoate, octanoates, decanoates or ricinoleates of alcohols or of polyalcohols, such as propylene glycol dioctanoate, cetyl octanoate, tridecyl octanoate, 2-ethylhexyl palmitate and 4-diheptanoate, alkyl benzoate, polyethylene glycol diheptanoate, propylene glycol di(2-ethylhexanoate) and their mixtures, C₁₂ to C₁₅ alkyl benzoates, hexyl laurate, esters of neopentanoic acid, such as isodecyl neopentanoate, isotridecyl neopentanoate, isostearyl neopentanoate or octyldodecyl neopentanoate, esters of isononanoic acid, such as isononyl isononanoate, isotridecyl isononanoate or octyl isononanoate, or hydroxylated esters, such as isostearyl lactate or diisostearyl malate;
esters of polyols and esters of pentaerythritol, such as dipentaerythritol tetrahydroxystearate/tetraisostearate,
fatty alcohols which are liquid at ambient temperature with a branched and/or unsaturated carbon chain having from 12 to 26 carbon atoms, such as 2-octyldodecanol, isostearyl alcohol, oleyl alcohol, 2-hexyldecanol, 2-butyloctanol and 2-undecylpentadecanol,
higher fatty acids, such as oleic acid, linoleic acid, linolenic acid and their mixtures, and
dialkyl carbonates, it being possible for the 2 alkyl chains to be identical or different, such as dicaprylyl carbonate, sold under the name Cetiol CC® by Cognis.

The nonvolatile silicone oils which can be used in the composition can be nonvolatile polydimethylsiloxanes (PDMSs), polydimethylsiloxanes comprising pendent alkyl or alkoxy groups and/or alkyl or alkoxy groups at the ends of the silicone chain, which groups each have from 2 to 24 carbon atoms, phenylated silicones, such as phenyl trimethicones, phenyl dimethicones, phenyl(trimethylsiloxy)diphenylsiloxanes, diphenyl dimethicones, diphenyl(methyldiphenyl)trisiloxanes and (2-phenylethyl)trimethylsiloxysilicates, dimethicones or phenyl trimethicones with a viscosity of less than or equal to 100 cSt, and their mixtures.

According to another embodiment, the silicone oil corresponds to the formula:

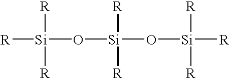

in which the R groups represent, independently of one another, a methyl or a phenyl. Preferably, in this formula, the organopolysiloxane comprises at least three phenyl groups, for example at least four or at least five.

Mixtures of the phenylated organopolysiloxanes described above can be used.

Mention may be made, for example, of mixtures of triphenylated, tetraphenylated or pentaphenylated organopolysiloxane.

According to another embodiment, the silicone oil corresponds to the formula:

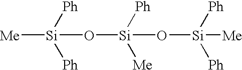

in which Me represents methyl and Ph represents phenyl. Such a phenylated silicone is manufactured in particular by Dow Corning under the reference Dow Corning 555 Cosmetic Fluid (INCI name: trimethyl pentaphenyl trisiloxane). The reference Dow Corning 554 Cosmetic Fluid can also be used.

The nonvolatile oil is preferably nonpolar, in the sense that its delta solubility a is equal to 0.

### Wax

The composition can comprise a wax. The term “wax”, within the meaning of the present invention, is understood to denote a lipophilic compound which is solid at ambient temperature (25° C.), which exhibits a reversible solid/liquid change in state and which has a melting point of greater than or equal to 30° C. which can reach up to 120° C.

The melting point of the wax can be measured using a differential scanning calorimeter (DSC), for example the calorimeter sold under the name DSC 30 by Mettler. The waxes can be hydrocarbon, fluorinated and/or silicone waxes. In particular, the waxes exhibit a melting point of greater than 25° C. and better still of greater than 45° C.

Mention may be made, as waxes which can be used in the composition, of linear hydrocarbon waxes. Their melting point is advantageously greater than 35° C., for example greater than 55° C. and preferably greater than 80° C.

The linear hydrocarbon waxes are advantageously chosen from substituted linear alkanes, unsubstituted linear alkanes, unsubstituted linear alkenes or substituted linear alkenes, an unsubstituted compound being composed solely of carbon and hydrogen. The substituents mentioned above not comprising carbon atoms.

The linear hydrocarbon waxes include polymers and copolymers of ethylene with a molecular weight of between 400 and 800, for example the Polywax 500 or Polywax 400 sold by New Phase Technologies.

The linear hydrocarbon waxes include linear paraffin waxes, such as the paraffin waxes S&P 206, S&P 173 and S&P 434 from Strahl & Pitsch.

The linear hydrocarbon waxes include long-chain linear alcohols, such as the products comprising a mixture of polyethylene and of alcohols comprising 20 to 50 carbon atoms, in particular the Performacol 425 or Performacol 550 (mixture in proportions 20/80) sold by New Phase Technologies.

Examples of silicone waxes are, for example:
the C₂₀₋₂₄ alkyl methicone, C₂₄₋₂₈ alkyl dimethicone, C₂₀₋₂₄ alkyl dimethicone and C₂₄₋₂₈ alkyl dimethicone sold by Archimica Fine Chemicals under the reference SilCare 41M40, SilCare 41M50, SilCare 41M70 and SilCare 41M80,
the stearyl dimethicones with the reference SilCare 41M65 sold by Archimica or with the reference DC-2503 sold by Dow Corning,
the stearoxytrimethylsilanes sold under the reference SilCare 1M71 or DC-580,
the products Abil Wax 9810, 9800 or 2440 from Wacker Chemie GmbH,
the C₃₀₋₄₅ alkyl methicones sold by Dow Corning under the reference AMS-C30 Wax and the C₃₀₋₄₅ alkyl dimethicones sold under the reference SF1642 or SF1632 by General Electric.

The amount of wax in the composition according to the invention can range from 5 to 70% by weight, with respect to the total weight of the composition, preferably from 5 to 40% by weight and better still from 10 to 30% by weight.

### Coloring Material

The composition according to the invention can comprise a colouring material in a proportion of, e.g., 0.5 to 50% of colouring material, preferably of 2 to 40% and better still of 5 to 30%, with respect to the total weight of the composition.

The colouring material can be any inorganic and/or organic compound exhibiting an absorption between 350 and 700 nm or capable of generating an optical effect, such as the reflection of incident light or interferences, for example.

The colouring materials of use in the present invention are chosen from all the organic and/or inorganic pigments known in the art, in particular those which are described in the Kirk-Othmer Encyclopaedia of Chemical Technology and in Ullmann's Encyclopaedia of Industrial Chemistry.

Mention may be made, as examples of inorganic colouring materials, of titanium dioxide, which is or is not surface treated, zinc oxide, zirconium or cerium oxides, iron or chromium oxides, manganese violet, ultramarine blue, chromium hydrate and ferric blue. For example, the following inorganic pigments can be used: Ta₂O₅, Ti₃O₅, Ti₂O₃, TiO, ZrO₂ as a mixture with TiO₂, ZrO₂, Nb₂O₅, CeO₂ or ZnS.

Mention may be made, as examples of organic colouring materials, of nitroso, nitro, azo, xanthene, quinoline, anthraquinone, phthalocyanine, of metal complex type, isoindolinone, isoindoline, quinacridone, perinone, perylene, diketopyrrolopyrrole, thioindigo, dioxazine, triphenylmethane or quinophthalone compounds.

In particular, the colouring materials can be chosen from carmine, carbon black, aniline black, azo yellow, quinacridone, phthalocyanine blue, sorghum red, the blue pigments classified in the Colour Index under the references CI 42090, 69800, 69825, 73000, 74100 and 74160, the yellow pigments classified in the Colour Index under the references CI 11680, 11710, 15985, 19140, 20040, 21100, 21108, 47000 and 47005, the green pigments classified in the Colour Index under the references CI 61565, 61570 and 74260, the orange pigments classified in the Colour Index under the references CI 11725, 15510, 45370 and 71105, the red pigments classified in the Colour Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915 and 75470, and the pigments obtained by oxidative polymerization of indole or phenol derivatives, as described in Patent FR 2 679 771.

The pigments in accordance with the invention can also be in the form of composite pigments, as described in Patent EP 1 184 426. These composite pigments can be composed in particular of particles comprising an inorganic core, at least one binder, which provides for the attachment of the organic pigments to the core, and at least one organic pigment at least partially covering the core.

The colouring materials can be chosen from dyes, lakes or pigments.

The dyes are, for example, fat-soluble dyes, although water-soluble dyes may be used. The fat-soluble dyes are, for example Sudan Red, D & C Red 17, D & C Green 6, β-carotene, soybean oil, Sudan Brown, D & C Yellow 11, D & C Violet 2, D & C Orange 5, quinoline yellow or annatto. They can represent from 0 to 20% of the weight of the composition and better still from 0.1 to 6%. The water-soluble dyes are in particular beetroot juice or methylene blue and can represent from 0.1 to 6% by weight of the composition (if present).

The term “lake” is understood to mean dyes adsorbed on insoluble particles, the combination thus obtained remaining insoluble when used. The inorganic substrates on which the dyes are adsorbed are, for example, alumina, silica, calcium sodium borosilicate, calcium aluminium borosilicate and aluminium. Mention may be made, among organic dyes, of cochineal carmine.

Mention may be made, as examples of lakes, of the products known under the following names: D & C Red 21 CI 45 380), D & C Orange 5 CI 45 370), D & C Red 27 CI 45 410), D & C Orange 10 CI 45 425), D & C Red 3 CI 45 430), D & C Red 7 (CI 15 850:1), D & C Red 4 (CI 15 510), D & C Red 33 (CI 17 200), D & C Yellow 5 (CI 19 140), D & C Yellow 6 (CI 15 985), D & C Green CI 61 570), D & C Yellow 10 CI 77 002), D & C Green 3 CI 42 053) or D & C Blue 1 CI 42 090).

The term “pigments” should be understood as meaning white or coloured and inorganic or organic particles intended to colour and/or opacify the composition. The pigments in accordance with the invention can, for example, be chosen from white or coloured pigments or from pigments possessing special effects, such as pearlescent agents, reflective pigments or interference pigments.

Mention may be made, as pigments which can be used in the invention, of titanium, zirconium or cerium oxides as well as zinc, iron or chromium oxides and ferric blue. Mention may be made, among the organic pigments which can be used in the invention, of carbon black and barium, strontium, calcium (D & C Red No. 7) and aluminium lakes.

The pearlescent agents can be present in the composition in a proportion of 0.001 to 20% of the total weight of the composition, preferably at a level of the order of 1 to 15%. Mention may be made, among the pearlescent agents which can be used in the invention, of mica covered with titanium oxide, with iron oxide, with natural pigment or with bismuth oxychloride, such as coloured titanium oxide-coated mica.

The pigments can be present in the composition in a proportion of 0.05 to 30% of the weight of the final composition and preferably in a proportion of 2 to 20%.

The variety of the pigments which can be used in the present invention makes it possible to obtain a rich palette of colours and also specific optical effects, such as metallic or interference effects.

The term “pigments possessing special effects” is understood to mean pigments which generally create a coloured appearance (characterized by a certain hue, a certain saturation and a certain lightness) which is non-uniform and which changes according to the conditions of observation (light, temperature, angles of observation, and the like). They consequently contrast with white or coloured pigments, which provide a conventional opaque, semitransparent or transparent uniform colouring.

Mention may be made, as examples of pigments possessing special effects, of white pearlescent pigments, such as mica covered with titanium dioxide or with bismuth oxychloride, coloured pearlescent pigments, such as mica covered with titanium dioxide and with iron oxides, mica covered with titanium dioxide and in particular with ferric blue or with chromium oxide or mica covered with titanium dioxide and with an organic pigment as defined above, and pearlescent pigments based on bismuth oxychloride. Mention may be made, as pearlescent pigments, of the following pearlescent agents, Cellini, sold by Engelhard (mica-TiO₂-lake), Prestige, sold by Eckart (mica-TiO₂), or Colorona, sold by Merck (mica-TiO₂—Fe₂O₃).

Mention may also be made of pigments possessing an interference effect which are not attached to a substrate, such as liquid crystals (Helicones HC from Wacker) or holographic interference flakes (Geometric Pigments or Spectra f/x from Spectratek). Pigments possessing special effects also comprise fluorescent pigments, whether they be substances which are fluorescent in daylight or which produce ultraviolet fluorescence, phosphorescent pigments, photochromic pigments and thermochromic pigments.

The composition advantageously comprises goniochromatic pigments, for example multilayer interference pigments, and/or reflective pigments. These two types of pigments are described in Application FR 0 209 246, the content of which is incorporated by reference in the present application.

The composition can comprise reflective pigments which may or may not be goniochromatic pigments and which may or may not be interference pigments.

Their size is compatible with the demonstration of a specular reflection of visible light (400-700 nm) of sufficient intensity, taking into account the mean gloss of the composition, to create a highlight point. This size is capable of varying according to the chemical nature of the particles, their shape and their capacity for specular reflection of visible light.

The reflective particles will preferably exhibit a dimension of at least 10 μm, for example of between approximately 20 μm and approximately 50 μm.

The term “dimension” denotes the dimension given by the statistical particle size distribution to half the population, referred to as D50. The size of the reflective particles can depend on their surface condition. The more reflective the latter, the smaller may a priori be the dimension, and vice versa.

Reflective particles usable in the invention, possessing a metallic or white glint, can, for example, reflect the light in all the components of the visible region without significantly absorbing one or more wavelengths. The spectral reflectance of these reflective particles can, for example, be greater than 70% within the 400-700 nm range and better still at least 80%, indeed even 90% or also 95%.

The reflective particles, whatever their shape, may or may not exhibit a multilayer structure and, in the case of a multilayer structure, may exhibit, for example, at least one layer of uniform thickness, in particular of a reflective material, which coats a substrate.

The substrate can be chosen from glasses, ceramics, graphite, metal oxides, aluminas, silicas, silicates, in particular aluminosilicates and borosilicates, and synthetic mica, this list not being limiting.

The reflective material can comprise a layer of metal or of a metal compound.

The layer of metal or of metal compound may or may not completely coat the substrate and the layer of metal may be at least partially covered with a layer of another material, for example a transparent material. It may be preferable for the layer of metal or of metal compound to completely coat the substrate, directly or indirectly, that is to say with insertion of at least one intermediate metal or non-metal layer.

The metal can be chosen, for example, from Ag, Au, Cu, Al, Ni, Sn, Mg, Cr, Mo, Ti, Pt, Va, Rb, W, Zn, Ge, Te, Se and their alloys. Ag, Au, Al, Zn, Ni, Mo, Cr, Cu and their alloys (for example, bronzes and brasses) are preferred metals.

In the case in particular of particles possessing a substrate coated with silver or with gold, the metal layer can be present at a content representing, for example, from 0.1 to 50% of the total weight of the particles, indeed even between 1 and 20%.

Particles of glass covered with a metal layer are described in particular in the documents JP-A-09188830, JP-A-10158450, JP-A-10158541, JP-A-07258460 and JP-A-05017710.

Particles possessing a glass substrate coated with silver, in the form of platelets, are sold under the name Microglass Metashine REFSX 2025 PS by Toyal. Particles possessing a glass substrate coated with nickel/chromium/molybdenum alloy are sold under the name Crystal Star GF 550 or GF 2525 by this same company.

The reflective particles, whatever their shape, can also be chosen from particles possessing a synthetic substrate at least partially coated with at least one layer of at least one metal compound, in particular a metal oxide, for example chosen from titanium oxides, in particular TiO₂, iron oxides, in particular Fe₂O₃, tin oxides, chromium oxides, barium sulphate and the following compounds: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅ MOS₂ and their mixtures or alloys.

Mention may be made, as examples of such particles, for example, of particles comprising a substrate of synthetic mica coated with titanium dioxide or particles of glass coated either, on the one hand, with brown iron oxide or, on the other hand, with titanium oxide, with tin oxide or with one of their mixtures, such as those sold under the Reflecks brand by Engelhard.

Pigments of the Metashine 1080R range, sold by Nippon Sheet Glass Co. Ltd., are also suitable for the invention. These pigments, more particularly described in Patent Application JP 2001-11340, are flakes of C-Glass glass comprising 65 to 72% of SiO₂ which are covered with a layer of titanium oxide of rutile (TiO₂) type. These glass flakes have a mean thickness of 1 micron and a mean size of 80 microns, i.e. a mean size/mean thickness ratio of 80. They exhibit blue, green, yellow or silver-coloured glints, depending on the thickness of the TiO₂ layer.

Mention may also be made of particles with a dimension of between 80 and 100 μm comprising a substrate of synthetic mica (fluorophlogopite) coated with titanium dioxide representing 12% of the total weight of the particle, these particles being sold under the name Prominence by Nihon Koken.

The reflective particles can also be chosen from particles formed of a stack of at least two layers possessing different refractive indices. These layers can be polymeric or metallic in nature and can in particular include at least one polymer layer. Such particles are described in particular in WO 99/36477, U.S. Pat. No. 6,299,979 and U.S. Pat. No. 6,387,498. Mention may be made, by way of illustration of the materials which can constitute the various layers of the multilayer structure, of, this list not being limiting: polyethylene naphthalate (PEN) and its isomers, poly(alkylene terephthalate)s and polyimides. Reflective particles comprising a stack of at least two layers of polymers are sold by 3M under the name Mirror Glitter. These particles comprise layers of 2,6-PEN and of poly(methyl methacrylate) in a ratio by weight of 80/20. Such particles are described in U.S. Pat. No. 5,825,643.

The composition can comprise one or more goniochromatic pigments.

The goniochromatic colouring agent can be chosen, for example, from multilayer interference structures and liquid crystal colouring agents.

In the case of a multilayer structure, the latter can comprise, for example, at least two layers, each layer, independently or not independently of the other layer(s), being produced, for example, from at least one material chosen from the group consisting of the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers and their combinations.

The multilayer structure may or may not exhibit, with respect to a central layer, a symmetry with regard to the chemical nature of the stacked layers.

Examples of symmetrical multilayer interference structures which can be used in are, for example, the following structures: Al/SiO₂/Al/SiO₂/Al, pigments having this structure being sold by DuPont de Nemours; Cr/MgF₂/Al/MgF₂/Cr, pigments having this structure being sold under the name Chromaflair by Flex; MOS₂/SiO₂/Al/SiO₂/MoS₂; Fe₂O₃/SiO₂/Al/SiO₂/Fe₂O₃ and Fe₂O₃/SiO₂/Fe₂O₃/SiO₂/Fe₂O₃, pigments having these structures being sold under the name Sicopearl by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂; SnO/TiO₂/SiO₂/TiO₂/SnO; Fe₂O₃/SiO₂/Fe₂O₃; SnO/mica/TiO₂/SiO₂/TiO₂/mica/SnO, pigments having these structures being sold under the name Xirona by Merck (Darmstadt). By way of example, these pigments can be pigments with a silica/titanium oxide/tin oxide structure sold under the name Xirona Magic by Merck, pigments with a silica/brown iron oxide structure sold under the name Xirona Indian Summer by Merck and pigments with a silica/titanium oxide/mica/tin oxide structure sold under the name Xirona Caribbean Blue by Merck. Mention may also be made of the Infinite Colors pigments from Shiseido. Different effects are obtained according to the thickness and the nature of the various layers. Thus, with the structure Fe₂O₃/SiO₂/Al/SiO₂/Fe₂O₃, the colour changes from green-golden to red-grey for SiO₂ layers of 320 to 350 nm; from red to golden for SiO₂ layers of 380 to 400 nm; from purple to green for SiO₂ layers of 410 to 420 nm; and from copper to red for SiO₂ layers of 430 to 440 nm.

Use may also be made of goniochromatic colouring agents possessing a multilayer structure comprising an alternation of polymer layers, for example of the polyethylene naphthalate and polyethylene terephthalate type. Such agents are described in particular in WO-A-96/19347 and WO-A-99/36478.

Mention may be made, as examples of pigments possessing a polymeric multilayer structure, of those sold by 3M under the name Color Glitter.

The liquid crystal colouring agents comprise, for example, silicones or cellulose ethers onto which mesomorphic groups are grafted.

Use may be made, as liquid crystal goniochromatic particles, for example, of those sold by Chenix and of those sold under the name Helicone® HC by Wacker.

The compositions according to the invention can be provided in any form acceptable and conventional for a cosmetic composition.

A person skilled in the art can choose the appropriate formulation form, and its method of preparation, on the basis of his general knowledge, taking into account, on the one hand, the nature of the constituents used, in particular their solubility in the support, and, on the other hand, the application envisaged for the composition.

The compositions in accordance with the invention can be used for caring for or making up keratinous substances, such as the hair, skin, eyelashes, eyebrows, nails, lips or scalp and more particularly for making up the lips, eyelashes and/or face.

They can thus be provided in the form of a product for caring for and/or making up the skin of the body or face, lips, eyelashes, eyebrows, hair, scalp or nails; of an antisun or self-tanning product; of a hair product, in particular for colouring, conditioning and/or caring for the hair; they are advantageously provided in the form of a mascara, lipstick, lip gloss, face powder, eyeshadow or foundation.

A further subject-matter of the invention is the use of two different polycondensates as described above, in particular in the proportions and the chemical compositions described above, for making up the lips in order to improve the fastness of the colour over time.

A further subject-matter of the invention is a method for the cosmetic treatment of keratinous substances, in particular the skin of the body or face, lips, nails, hair and/or eyelashes, comprising the application, to the materials, of a cosmetic composition as defined above.

This method according to the invention makes it possible in particular to care for or make up the lips by application of a lipstick or lip gloss composition according to the invention.

The invention also relates to the use of the compositions described above for making up the lips. Another subject-matter of the present invention is a cosmetic combination comprising:
a container delimiting at least one compartment, the container being closed by a closing element; and
a composition as described above positioned inside the compartment.

The container can have any appropriate form. It can in particular be in the form of a bottle, a tube, a pot, a box, a tin, a bag or a case.

The closing element can be in the form of a removable stopper, of a lid, of a seal, of a tear-off strip or of a capsule, in particular of the type comprising a body fixed to the container and a cap articulated over the body. It can also be in the form of an element providing the selective closure of the container, in particular a pump, a valve or a flap.

The container can be used in combination with an applicator, in particular in the form of a brush comprising an arrangement of hairs held by a twisted wire. Such a twisted brush is described in particular in U.S. Pat. No. 4,887,622. It can also be in the form of a comb comprising a plurality of application elements, obtained in particular from moulding. Such combs are described, for example, in Patent FR 2 796 529. The applicator can be in the form of a fine brush, such as described, for example, in Patent FR 2 722 380. The applicator can be in the form of a pad of foam or elastomer, of a felt-tipped pen or of a spatula. The applicator can be free (powder puff or sponge) or integrally attached to a rod carried by the closing element, such as described, for example, in U.S. Pat. No. 5,492,426. The applicator can be integrally attached to the container, such as described, for example, in Patent FR 2 761 959.

The product may be contained directly in the container or indirectly. By way of example, the product can be positioned on an impregnated support, particularly in the form of a wipe or of a wad, and can be positioned (singly or severally) in a tin or in a bag. Such a support incorporating the product is described, for example, in Application WO 01/03538.

The closing element can be coupled to the container by screwing. Alternatively, the coupling between the closing element and the container is carried out other than by screwing, in particular via a bayonet mechanism, by snapping, clamping, welding or adhesive bonding, or by magnetic attraction. The term “snapping” is understood to mean in particular any system involving the crossing of a row or strip of material by elastic deformation of a portion, in particular of the closing element, and then by elastically returning the portion to the unstressed position after the row or strip has been crossed.

The container can be at least partially made of thermoplastic material. Mention may be made, as examples of thermoplastic materials, of polypropylene or polyethylene.

Alternatively, the container is made of non-thermoplastic material, in particular of glass or of metal (or alloy).

The container can have rigid walls or deformable walls, in particular in the form of a tube or of a tube bottle.

The container can comprise means intended to bring about or facilitate the distribution of the composition. By way of example, the container can have deformable walls, so as to bring about the departure of the composition in response to excess pressurization inside the container, which excess pressurization is brought about by the elastic (or non-elastic) crushing of the walls of the container. Alternatively, in particular when the product is in the form of a stick, the latter can be driven by a piston mechanism. Still in the case of a stick, in particular of a make-up product (lipstick, foundation, and the like), the container can comprise a mechanism, in particular a rack-and-pinion mechanism or a mechanism with a screw rod or a mechanism with a helical groove, capable of moving a stick in the direction of the opening. Such a mechanism is described, for example, in Patent FR 2 806 273 or in Patent FR 2 775 566. Such a mechanism for a liquid product is described in Patent FR 2 727 609.

The container can be composed of a case with a bottom delimiting at least one receptacle comprising the composition and a lid, in particular articulated over the bottom, capable of at least partially covering the bottom. Such a case is described, for example, in Application WO 03/018423 or in Patent FR 2 791 042.

The container can be equipped with a drainer positioned in the vicinity of the opening of the container. Such a drainer makes it possible to wipe the applicator and optionally the rod to which it may be integrally attached. Such a drainer is described, for example, in Patent FR 2 792 618.

The composition can be at atmospheric pressure inside the container (at ambient temperature) or pressurized, in particular using a propellant gas (aerosol). In the latter case, the container is equipped with a valve (of the type of those used for aerosols).

The invention is illustrated in more detail in the following examples.

### Method for Measuring the Viscosity

The viscosity at 80° C. or at 110° C. of the polymer is measured using a cone/plate viscometer of Brookfield CAP 1000+type.

The appropriate cone/plate is determined by a person skilled in the art on the basis of his knowledge; in particular:
between 50 and 500 mPa·s, use is made of a cone O₂
between 500 and 1000 mPa·s: cone 03
between 1000 and 4000 mPa·s: cone 05
between 4000 and 10 000 mPa·s: cone 06

### Example 1

### Synthesis of pentaerythrityl benzoate/isophthalate/isostearate

20 g of benzoic acid, 280 g of isostearic acid and 100 g of pentaerythritol are charged to a reactor equipped with a mechanical stirrer, an argon inlet and a distillation system and then the mixture is gradually heated, under a gentle argon stream, to 110-130° C. in order to obtain a homogeneous solution. The temperature is subsequently gradually increased up to 180° C. and this temperature is maintained for approximately 2 hours. The temperature is again increased up to 220° C. and this temperature is maintained until an acid number of less than or equal to 1 is obtained, which takes approximately 11 hours. The mixture is cooled to a temperature of between 100 and 130° C., then 100 g of isophthalic acid are introduced and the mixture is again gradually heated up to 220° C. for approximately 11 hours.

405 g of pentaerythrityl benzoate/isophthalate/isostearate polycondensate are thus obtained in the form of a very thick oil.

The polycondensate exhibits the following characteristics:
soluble to 50% by weight, at 25° C., in Parleam
acid number=3.7
hydroxyl number=72
Mw=59 400
η_{110° C.}=1510 mPa·s
ratio of the number of moles of aromatic monocarboxylic acid to the number of moles of nonaromatic branched monocarboxylic acid: 0.16.

### Example 2

### Synthesis of pentaerythrityl benzoate/isophthalate/isostearate

35 g of benzoic acid, 270 g of isostearic acid and 80 g of pentaerythritol are charged to a reactor equipped with a mechanical stirrer, an argon inlet and a distillation system and then the mixture is gradually heated, under a gentle argon stream, to 110-130° C. in order to obtain a homogeneous solution. The temperature is subsequently gradually increased up to 180° C. and this temperature is maintained for approximately 2 hours. The temperature is again increased up to 220° C. and this temperature is maintained until an acid number of less than or equal to 1 is obtained, which takes approximately 11 hours. The mixture is cooled to a temperature of between 100 and 130° C., then 65 g of isophthalic acid are introduced and the mixture is again gradually heated up to 220° C. for approximately 5 hours.

380 g of pentaerythrityl benzoate/isophthalate/isostearate polycondensate are thus obtained in the form of an oil.

The polycondensate exhibits the following characteristics:
soluble to 50% by weight, at 25° C., in Parleam
acid number=5.5
hydroxyl number=103
Mw=7200
η_{80° C.}=700 mPa·s
ratio of the number of moles of aromatic monocarboxylic acid to the number of moles of nonaromatic branched monocarboxylic acid: 0.30.

### Example 3

### Synthesis of pentaerythrityl benzoate/isophthalate/stearate

10 g of benzoic acid, 370 g of stearic acid and 95 g of pentaerythritol are charged to a reactor equipped with a mechanical stirrer, an argon inlet and a distillation system and then the mixture is gradually heated, under a gentle argon stream, to 110-130° C. in order to obtain a homogeneous solution. The temperature is subsequently gradually increased up to 180° C. and this temperature is maintained for approximately 2 hours. The temperature is again increased up to 220° C. and this temperature is maintained until an acid number of less than or equal to 1 is obtained, which takes approximately 11 hours. The mixture is cooled to a temperature of between 100 and 130° C., then 90 g of isophthalic acid are introduced and the mixture is again gradually heated up to 220° C. for approximately 11 hours.

430 g of pentaerythrityl benzoate/isophthalate/stearate polycondensate are thus obtained in the form of a very thick oil.

The polycondensate exhibits the following characteristics:
soluble to 50% by weight, at 70° C., in Parleam
acid number=10.8
Mw=8800
η_{80° C.}=360 mPa·s

### Examples A to R

The following polycondensates are prepared in a similar way to the preceding examples (the % values are by weight):

| | | | Poly- | | |
|---|---|---|---|---|---|
| | | | carboxylic | | |
| | | Aromatic | acid or | | |
| | | acid (% | anhydride | Nonaromatic | |
| | Polyol (% | and | (% and | acid (% and | |
| | and nature) | nature) | nature) | nature) | Solubility* |
| Example | 21.6 | 3.9 | 19.5 | 27.5% iso- | at 25° C. |
| A | penta- | benzoic | isophthalic | stearic + | |
| | erythritol | | acid | 27.5% | |
| | | | | isononanoic | |
| Example | 16.8 | 1.8 | 15.9 | 65.5 | at 70° C. |
| B | penta- | benzoic | isophthalic | behenic | |
| | erythritol | | acid | | |
| Example | 20 | 4 | 20 | 56 | at 25° C. |
| C | penta- | (tert- | isophthalic | isostearic | |
| | erythritol | butyl)- | acid | | |
| | | benzoic | | | |
| Example | 17.4 | 8.6 | 16 | 58 | at 25° C. |
| D | glycerol | benzoic | isophthalic | isostearic | |
| | | | acid | | |
| Example | 20.7 | 8.5 | 15.9 | 54.9 | at 25° C. |
| E | glycerol | (tert- | adipic acid | isononanoic | |
| | | butyl)- | | | |
| | | benzoic | | | |
| Example | 25.5 | 2 | 13.7 | 58.8 | at 25° C. |
| F | diglycerol | benzoic | isophthalic | isononanoic | |
| | | | acid | | |
| Example | 28 | 2 | 14 | 56 | at 25° C. |
| G | ditrimethylol- | 1-naphthoic | isophthalic | isostearic | |
| | propane | | acid | | |
| Example | 25.2 | 5.8 | 12.6 | 56.3 | at 25° C. |
| H | trimethylol- | benzoic | isophthalic | isononanoic | |
| | propane | | acid | | |
| Example | 25 | 2.1 | 14.6 | 58.3 | at 25° C. |
| I | trimethylol- | m-toluic | phthalic | isostearic | |
| | propane | | anhydride | | |
| Example | 21.9 | 6.3 | 13.5 | 58.3 | at 25° C. |
| J | erythritol | (tert- | sebacic | isooctanoic | |
| | | butyl)- | acid | acid | |
| | | benzoic | | | |
| Example | 20.4 | 6.1 | 20.4 | 53.1 | at 25° C. |
| K | dipenta- | benzoic | Pripol | isostearic | |
| | erythritol | | 1009** | | |
| Example | 28 | 2 | 14 | 40% iso- | at 25° C. |
| L | ditrimethylol- | 1-naphthoic | isophthalic | stearic + | |
| | propane | | acid | 16% | |
| | | | | 2-ethyl- | |
| | | | | hexanoic | |
| Example | 21.3 | 6.4 | 17 | 27.7% | at 25° C. |
| M | penta- | benzoic | succinic | nonanoic + | |
| | erythritol | | acid | 27.6% | |
| | | | | iso- | |
| | | | | heptanoic | |
| Example | 17.4 | 8.6 | 16 | 58 | at 70° C. |
| N | glycerol | benzoic | isophthalic | stearic | |
| | | | acid | | |
| Example | 25.5 | 2 | 13.7 | 58.8 | at 70° C. |
| O | diglycerol | benzoic | isophthalic | myristic | |
| | | | acid | | |
| Example | 25.5 | 3.9 | 15.7 | 54.9 | at 70° |
| P | diglycerol | benzoic | sebacic | lauric | |
| | | | acid | | |
| Example | 20.4 | 6.1 | 20.4 | 53.1 | at 70° C. |
| Q | dipenta- | benzoic | Pripol | behenic | |
| | erythritol | | 1009** | | |
| Example | 25.2 | 5.8 | 12.6 | 31.1% | at 70° C. |
| R | trimethylol- | benzoic | isophthalic | stearic + | |
| | propane | | acid | 25.3% | |
| | | | | behenic | |

*“at 25° C.” indicates that the polymer is soluble to 50% by weight, at 25° C., in Parleam; “at 70° C.” indicates that the polymer is soluble to 50% by weight, at 70° C., in Parleam
**Pripol 1009 from Uniqema: oleic acid dimer

### Example 4

### Synthesis of pentaerythrityl benzoate/isophthalate/isostearate/stearate

20 g of benzoic acid, 210 g of stearic acid, 70 g of isostearic acid and 100 g of pentaerythritol are charged to a reactor equipped with a mechanical stirrer, an argon inlet and a distillation system and then the mixture is gradually heated, under a gentle argon stream, to 110-130° C. in order to obtain a homogeneous solution. The temperature is subsequently gradually increased up to 180° C. and this temperature is maintained for approximately 2 hours. The temperature is again increased up to 220° C. and this temperature is maintained until an acid number of less than or equal to 1 is obtained, which takes approximately 11 hours. The mixture is cooled to a temperature of between 100 and 130° C., then 100 g of isophthalic acid are introduced and the mixture is again gradually heated up to 220° C. for approximately 11 hours.

450 g of pentaerythrityl benzoate/isophthalate/isostearate/stearate polycondensate are thus obtained in the form of a very thick oil.

The polycondensate exhibits the following characteristics:
soluble to 50% by weight, at 70° C., in Parleam
acid number=7.1
η_{110° C.}=850 mPa·s
Mw=28 500
ratio of the number of moles of aromatic monocarboxylic acid to the number of moles of nonaromatic monocarboxylic acids: 0.166.

### Example 5

### Synthesis of pentaerythrityl behenate/benzoate/isophthalate/isostearate

20 g of benzoic acid, 140 g of behenic acid, 140 g of isostearic acid and 100 g of pentaerythritol are charged to a reactor equipped with a mechanical stirrer, an argon inlet and a distillation system and then the mixture is gradually heated, under a gentle argon stream, to 110-130° C. in order to obtain a homogeneous solution. The temperature is subsequently gradually increased up to 180° C. and this temperature is maintained for approximately 2 hours. The temperature is again increased up to 220° C. and this temperature is maintained until an acid number of less than or equal to 1 is obtained, which takes approximately 11 hours. The mixture is cooled to a temperature of between 100 and 130° C., then 100 g of isophthalic acid are introduced and the mixture is again gradually heated up to 220° C. for approximately 11 hours.

440 g of pentaerythrityl behenate/benzoate/isophthalate/isostearate polycondensate are thus obtained in the form of a very thick oil.

The polycondensate exhibits the following characteristics:
soluble to 50′ by weight, at 70° C., in Parleam
acid number=4.2
η_{110° C.}=2050 mPa·s
ratio of the number of moles of aromatic monocarboxylic acid to the number of moles of nonaromatic monocarboxylic acids: 0.181.

### Examples a to j

The following polycondensates are prepared in a similar way to the preceding examples (the % values are by weight):

| | | | Poly- | | |
|---|---|---|---|---|---|
| | | | carboxylic | | |
| | | | acid or | | |
| | | Aromatic | anhydride | Nonaromatic | |
| | Polyol (% | acid (% and | (% and | acids (% | |
| | and nature) | nature) | nature) | and nature) | Solubility* |
| Example | 20.4 | 4.1 | 18.3 | 28.6% iso- | at 25° C. |
| a | penta- | benzoic | isophthalic | stearic + | |
| | erythritol | | acid | 14.3% | |
| | | | | isononanoic + | |
| | | | | 14.3% | |
| | | | | stearic | |
| Example | 20 | 4 | 20 | 18% iso- | at 25° C. |
| b | penta- | benzoic | isophthalic | stearic + | |
| | erythritol | | acid | 38% stearic | |
| Example | 20 | 4 | 20 | 28% iso- | at 25° C. |
| c | penta- | benzoic | isophthalic | stearic + | |
| | erythritol | | acid | 28% stearic | |
| Example | 19.8 | 4 | 19.8 | 40.6% iso- | at 25° C. |
| d | penta- | benzoic | isophthalic | stearic + | |
| | erythritol | | acid | 15.8% | |
| | | | | stearic | |
| Example | 19.8 | 4 | 19.8 | 48.5% iso- | at 25° C. |
| e | penta- | benzoic | isophthalic | stearic + | |
| | erythritol | | acid | 7.9% | |
| | | | | stearic | |
| Example | 19.8 | 4 | 19.8 | 52.4% iso- | at 25° C. |
| f | penta- | benzoic | isophthalic | stearic + | |
| | erythritol | | acid | 4% stearic | |
| Example | 25.5 | 3.9 | 15.7 | 34.9% iso- | at 25° C. |
| g | diglycerol | benzoic | sebacic | stearic + | |
| | | | acid | 20% lauric | |
| Example | 25 | 2.1 | 14.6 | 18.3% iso- | at 70° C. |
| h | trimethylol- | m-toluic | phthalic | stearic + | |
| | propane | | anhydride | 40% behenic | |
| Example | 21.9 | 6.3 | 13.5 | 8.3% iso- | at 70° C. |
| i | erythritol | (tert- | sebacic | octanoic + | |
| | | butyl)- | acid | 50% stearic | |
| | | benzoic | | | |
| Example | 20.7 | 8.5 | 15.9 | 45.9% iso- | at 25° C. |
| j | glycerol | (tert- | adipic acid | nonanoic + | |
| | | butyl)- | | 9% behenic | |
| | | benzoic | | | |

*“at 25° C.” indicates that the polymer is soluble to 50% by weight, at 25° C., in Parleam; “at 70° C.” indicates that the polymer is soluble to 50% by weight, at 70° C., in Parleam.

### Example 6

### Synthesis of pentaerythrityl benzoate/isophthalate/laurate/PDMS

150 g of benzoic acid, 165 g of lauric acid and 110 g of pentaerythritol are charged to a reactor equipped with a mechanical stirrer, an argon inlet and a distillation system and then the mixture is gradually heated, under a gentle argon stream, to 110-130° C. in order to obtain a homogeneous solution. The temperature is subsequently gradually increased up to 180° C. and this temperature is maintained for approximately 2 hours. The temperature is again increased up to 220° C. and this temperature is maintained until an acid number of less than or equal to 1 is obtained, which takes approximately 15 hours. The mixture is cooled to a temperature of between 100 and 130° C., 90 g of isophthalic acid and 50 g of α,ω-dihydroxysilicone X22-160AS from Shin-Etsu are then introduced and the mixture is again gradually heated up to 220° C. for approximately 11 hours.

510 g of pentaerythrityl benzoate/isophthalate/laurate/PDMS polycondensate are thus obtained in the form of a thick oil which solidifies at ambient temperature.

The polycondensate exhibits the following characteristics:
acid number=28.7
hydroxyl number=85
η_{110° C.}=2.1 poises (i.e. 210 mPa·s)
ratio of the number of moles of aromatic monocarboxylic acid to the number of moles of nonaromatic branched monocarboxylic acid: 1.49.

500 g of polycondensate obtained above are withdrawn and heated to 70° C., 215 g of ethyl acetate are slowly run in with stirring and then clarification is carried out by filtering under hot conditions through a sintered glass No. 2 funnel. After cooling to ambient temperature, 705 g of a 70% solution of polycondensate in ethyl acetate are obtained, the solution existing in the form of a viscous pale-yellow liquid having a viscosity at 25° C. of approximately 165 centipoises (mPa·s).

### Example 7

### Synthesis of pentaerythrityl benzoate/isophthalate/laurate

165 g of benzoic acid, 160 g of lauric acid and 120 g of pentaerythritol are charged to a reactor equipped with a mechanical stirrer, an argon inlet and a distillation system and then the mixture is gradually heated, under a gentle argon stream, to 110-130° C. in order to obtain a homogeneous solution. The temperature is subsequently gradually increased up to 180° C. and this temperature is maintained for approximately 2 hours. The temperature is again increased up to 220° C. and this temperature is maintained until an acid number of less than or equal to 1 is obtained, which takes approximately 15 hours. The mixture is cooled to a temperature of between 100 and 130° C., 100 g of isophthalic acid and are then introduced and the mixture is again gradually heated up to 220° C. for approximately 12 hours.

510 g of pentaerythrityl benzoate/isophthalate/laurate polycondensate are thus obtained in the form of a thick oil which solidifies at ambient temperature.

The polycondensate exhibits the following characteristics:
acid number=20.4
hydroxyl number=66
η_{110° C.}=4.7 poises (i.e. 470 mPa·s)
ratio of the number of moles of aromatic monocarboxylic acid to the number of moles of nonaromatic branched monocarboxylic acid: 1.69.

500 g of polycondensate obtained above are withdrawn and heated to 70° C., 215 g of ethyl acetate are slowly run in with stirring and then clarification is carried out by filtering under hot conditions through a sintered glass No. 2 funnel. After cooling to ambient temperature, 700 g of a 70% solution of polycondensate in ethyl acetate are obtained, the solution existing in the form of a viscous pale-yellow liquid having a viscosity at 25° C. of approximately 310 centipoises (mPa·s).

### Example 8

### Synthesis of pentaerythrityl benzoate/phthalate/laurate

185 g of benzoic acid, 174 g of lauric acid and 114.6 g of pentaerythritol are charged to a reactor equipped with a mechanical stirrer, an argon inlet and a distillation system and then the mixture is gradually heated, under a gentle argon stream, to 110-130° C. in order to obtain a homogeneous solution. The temperature is subsequently gradually increased up to 180° C. and this temperature is maintained for approximately 2 hours. The temperature is again increased up to 220° C. and this temperature is maintained until an acid number of less than or equal to 1 is obtained, which takes approximately 18 hours. The mixture is cooled to a temperature of between 100 and 130° C., 80 g of phthalic anhydride are then introduced and the mixture is again gradually heated up to 220° C. for approximately 8 hours. 15 g of pentaerythritol are added and the mixture is maintained at 220° C. for 8 hours. 512 g of pentaerythrityl benzoate/phthalate/laurate polycondensate are thus obtained in the form of a thick oil which solidifies at ambient temperature.

The polycondensate exhibits the following characteristics:
acid number=13.0
hydroxyl number=60
η_{110° C.}=0.9 poises (i.e. 90 mPa·s)
ratio of the number of moles of aromatic monocarboxylic acid to the number of moles of nonaromatic branched monocarboxylic acid: 1.74.

### Example 9 of a Stick of Lipstick

| | |
|---|---|
| Ingredient (INCI name) | % W |

| | | |
|---|---|---|
| A | Trimethyl pentaphenyl trisiloxane | 57.55 |
| | Polycondensate of Example 1 | 16.00 |
| B | Microcrystalline wax | 4.55 |
| | Beeswax | 1.95 |
| C | Polycondensate of Example 3 | 1.00 |
| | Bis-diglyceryl polyacyladipate-2 | 12 |
| D | Rutile titanium oxide treated with | 0.20 |
| | alumina/silica/trimethylolpropane | |
| | Aluminium lake of Brilliant Blue FCF on | 0.20 |
| | alumina | |
| | Brown, yellow iron oxides | 0.95 |
| | Aluminium lake of tartrazine on alumina | 0.85 |
| | Calcium salt of Lithol Red B | 0.45 |
| E | Titanium oxide-coated mica | 2.80 |
| | Titanium oxide-coated mica | 1.00 |
| | Titanium oxide-coated mica | 0.50 |

| | |
|---|---|
| Total | 100.00 |

The above written description of the invention provides a manner and process of making and using it such that any person skilled in this art is enabled to make and use the same, this enablement being provided in particular for the subject matter of the appended claims, which make up a part of the original description and including a composition, comprising:
at least one first polyester capable of being obtained by reaction:
   of at least one polyol comprising 3 to 6 hydroxyl groups;
   of at least one nonaromatic branched monocarboxylic acid;
   of at least one aromatic monocarboxylic acid, and
   of at least one polycarboxylic acid comprising at least 2 carboxyl groups COOH and/or one cyclic anhydride of such a polycarboxylic acid,
at least one second polyester capable of being obtained by reaction:
   of at least one polyol comprising 3 to 6 hydroxyl groups;
   of at least one nonaromatic linear monocarboxylic acid;
   of at least one aromatic monocarboxylic acid, and
   of at least one polycarboxylic acid comprising at least 2 carboxyl groups COOH and/or one cyclic anhydride of such a polycarboxylic acid.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. A composition, comprising:
at least one first polyester obtained by reaction:
of at least one polyol comprising 3 to 6 hydroxyl groups;
of at least one nonaromatic branched monocarboxylic acid;
of at least one aromatic monocarboxylic acid, and
of at least one polycarboxylic acid comprising at least 2 carboxyl groups COOH and/or one cyclic anhydride of such a polycarboxylic acid,
at least one second polyester different from said first polyester obtained by reaction:
of at least one polyol comprising 3 to 6 hydroxyl groups;
of at least one nonaromatic linear monocarboxylic acid;
of at least one aromatic monocarboxylic acid, and
of at least one polycarboxylic acid comprising at least 2 carboxyl groups COOH and/or one cyclic anhydride of such a polycarboxylic acid.

2. The composition according to claim 1, wherein the polyol of the first and/or second polyester is chosen from glycerol, pentaerythritol, diglycerol, sorbitol and their mixtures.

3. The composition according to claim 1, wherein the nonaromatic branched monocarboxylic acid of the first and/or second polyester is chosen from 2-ethylhexanoic acid, isooctanoic acid, isoheptanoic acid, isononanoic acid, isostearic acid and their mixtures.

4. The composition according to claim 1, wherein the nonaromatic linear monocarboxylic acid of the first and/or second polyester is chosen from lauric acid, myristic acid, nonanoic acid, palmitic acid, stearic acid, behenic acid and their mixtures.

5. The composition according to claim 1, wherein the aromatic monocarboxylic acid of the first and/or second polyester is chosen from benzoic acid, 4-(tert-butyl)benzoic acid, o-toluic acid, m-toluic acid 1-naphthoic acid, and their mixtures.

6. The composition according to claim 1, wherein the aromatic monocarboxylic acid of the first and/or second polyester represents 0.5 to 9.95% by weight of the total weight of the final polyester.

7. The composition according to claim 1, wherein the polycarboxylic acid of the first and/or second polyester is chosen from saturated or unsaturated, indeed even aromatic, and linear, branched and/or cyclic polycarboxylic acids comprising 2 to 50 carbon atoms, the acid comprising at least two carboxyl COOH groups.

8. The composition according to claim 1, wherein the polycarboxylic acid or its anhydride of the first and/or second polyester is chosen from adipic acid, phthalic anhydride and/or isophthalic acid.

9. The composition according to claim 1, wherein the polycarboxylic acid and/or its cyclic anhydride of the first and/or second polyester represents 10 to 30% by weight of the total weight of the polyester.

10. The composition according to claim 1, wherein the first and second polyesters are obtained by reaction:
of at least one polyol chosen, alone or as a mixture, from glycerol, pentaerythritol, sorbitol and their mixtures; present in an amount of 10 to 30% by weight with respect to the total weight of the final polyester;
of at least one nonaromatic monocarboxylic acid; present in an amount of 30 to 80% by weight with respect to the total weight of the final polyester;
of at least one aromatic monocarboxylic acid chosen, alone or as a mixture, from benzoic acid, o-toluic acid, m-toluic acid or 1-naphthoic acid; present in an amount of 0.1 to 10% by weight with respect to the total weight of the final polyester; and
of at least one polycarboxylic acid or one of its anhydrides chosen, alone or as a mixture, from phthalic anhydride and isophthalic acid; present in an amount of 5 to 40% by weight with respect to the total weight of the final polyester.

11. The composition according to claim 1, wherein the first polyester is present in a total amount of between 1 and 50% by weight with respect to the weight of the composition.

12. The composition according to claim 1, wherein the second polyester is present in a total amount of between 0.1 and 20% by weight with respect to the weight of the composition.

13. A composition, comprising:
a first benzoic acid/isophthalic acid/isostearic acid/pentaerythritol polyester, and
a second benzoic acid/isophthalic acid/stearic acid/pentaerythritol polyester.

14. The composition according to claim 1, wherein the first polymer and the second polymer are present in a ratio by weight of 50/1-2/1.

15. The composition according to claim 1, wherein said composition further comprises at least one of a wax, a colouring material, and a nonvolatile oil.

16. A method, comprising applying the composition of claim 1 to the skin, lips, hair, eyelashes, eyebrows or nails.

17. A method, comprising applying the composition of claim 15 to the skin, lips, hair, eyelashes, eyebrows or nails.
